(19) United States
(12) Patent Application Publication
(10) Pub. No.: US 2010/0062078 A1
(43) Pub. Date: Mar. 11, 2010

(54) PHARMACEUTICAL COMPOSITION FOR TREATING CANCER AND THE SIGNALING PATHWAY THEREOF

(75) Inventors: Shyng-Shiou Yuan, Kaohsiung City (TW); Yang-Chang Wu, Kaohsiung City (TW); Hsueh-Ling Chang, Kaohsiung City (TW); Fang-Rong Chang, Kaohsiung City (TW)

Correspondence Address: VOLPE AND KOENIG, P.C., UNITED PLAZA, SUITE 1600, 30 SOUTH 17TH STREET, PHILADELPHIA, PA, 19103, US

(73) Assignees: Shyng-Shiou Yuan, Kaohsiung City (TW); Yang-Chang Wu, Kaohsiung City (TW)

(21) Appl. No.: 12/399,155

(22) Filed: Mar. 6, 2009

(30) Foreign Application Priority Data
Sep. 5, 2008 (TW) 097134287

(51) Int. Cl. A61K 31/352; C07D 311/22; A61K 33/24; A61P 35/00

(52) U.S. Cl. 424/649; 549/403; 514/456

(57) ABSTRACT

A pharmaceutical composition, composed of protoapigenone (formula I), for treating gynecological cancers, prostate cancer, urinary bladder cancer and hepatocarcinoma is provided. Protoapigenone has cytotoxicity on these cancers, and arrests the cell cycle at S and G2/M phases. In addition, protoapigenone regulates cell signaling pathways, such as caspase-3, PARP, p-38 MAPK and JNK½, to induce apoptosis. Protoapigenone significantly inhibits the xenograft tumor growth on nude mice, without major side effects. Co-treatment o protoapigenone of protoapigenone and cisplatin shows synergistic cytotoxicity of MDAH-2774 ovarian cancer cells.

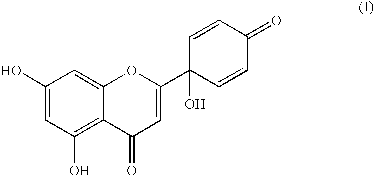

### FIELD OF THE INVENTION

The present invention relates to a pharmaceutical composition composed of flavonoid compound. In particular, the present invention relates to a pharmaceutical composition composed of a novel flavonoid, protoapigenone, for treating cancer such as gynecological cancers and prostate cancer, etc. and relates to the research on the signaling pathways of cancer cells induced by the pharmaceutical composition.

### BACKGROUND OF THE INVENTION

Flavonoid or bioflavonoid, a polyphenolic compound, can inhibit human cancer cell growth. Flavonoid has a structure of phenylbenzopyrone (C6-C3-C6), mainly including flavone, flavanol, isoflavone, flavonol, flavanone and flavanonol (Ren et al., 2003). The above-mentioned flavonoids can be found in some dietary plants, some medical plants and herbal remedies (Kajimoto et al., 2002; Samuelsen, 2000). Some flavonoids such as apigenin, genistein and catechin are proved to own the growth inhibition on ovarian cancer, breast cancer, colon cancer and leukemia (Birt et al., 2001; Brusselmans et al., 2005; Fang et al., 2007; Friedman et al., 2007; Gossner et al., 2007; Hamblin, 2006; Seo et al., 2006; Spinella et al., 2006). The bioactivity of flavonoid includes apoptosis induction, cell cycle arrest, growth inhibition, and angiogenesis inhibition, antioxidation and the combination thereof (Ren et al., 2003). These bioactivities is accomplished by regulating the signaling transduction pathways, such as nuclear factor-κB (NFκB), activator protein-1 or mitogen-activated protein kinases (MAPKs) (Fresco et al., 2006; Kong et al., 2001; Sarkar and Li, 2004). This renders that falvonoid might be the effective anticancer agent.

Protoapigenone, a novel flavonoid, has a structure as formula I and is extracted from the whole plant Thelypteris torresiana (Gaud), a native fern in Taiwan. The preparation method of protoapigenone was disclosed in Taiwan Patent Application No. 094139201. In addition, Lin et al. (2005) has proved that protoapigenone has cytotoxicity on human liver cancer cell lines Hep G2 and Hep 3B, human breast adenocarcinoma cell line MCF-7, human lung adenocarcinoma epithelial cell line A549 and human breast cancer cell line MDA-MB-231. However, the literature does not disclose whether protoapigenone can inhibit growth of other cancer cell lines and the signaling pathways thereof. Simultaneously, one skilled in the art also cannot infer that protoapigenone also can inhibit the growth of other cancer cell lines and has other bioactivities from the applications of protoapigenone on the abovementioned cancers.

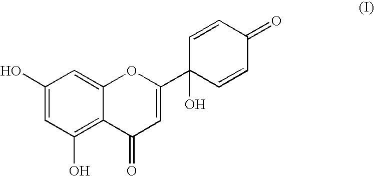

Gynecological cancer is a cancer originated from female reproductive organs, such as cervix, fallopian tubes, ovaries, uterus, vagina and vulva. Half of eighty thousand new diagnosed cases in the United States are uterus cancer. The risk to suffer cancer increases along with ages, and genetic mutation or family inheritance also increases the risk. Paclitaxel (Taxol®) and paraplatin (Carboplatin®) have good early therapeutic effect in the later-stage ovarian cancer which has a remission rate of 60% to 80%. However, the recurrence is relatively high. Finally, up to 70% patients die.

Prostate cancer is another common carcinoma and is ranked in the third one of the female cancer-related death causes in the United States (Jemal et al., 2006). Surgery and radiotherapy are the major curative methods for low-to-moderately differentiated prostate cancer (Kish et al., 2001). Once the cancer cells spreads beyond the pelvis, there is no effective cure for the prostate cancer, and the treatment relies heavily on the chemotherapy to control the cancer growth. However, the clinically used chemotherapeutic agents remain highly toxic to normal tissues (Moss and Petrylak, 2006).

Therefore, to resolve the drug problems of gynecological cancers and prostate cancer, and the difficulty of newly synthetic drugs by chemosynthesis being overcome by the natural drugs extracted from the plant, flavonoid becomes a new choice for the new drug development.

It is therefore attempted by the applicant to deal with the above situation encountered in the prior art.

### SUMMARY OF THE INVENTION

In order to the gynecological cancer and the prostate cancer treatments, the pharmaceutical composition composed of a flavonoid compound is extracted from the fern, and is performed in vitro and in vivo experiments on the gynecological cancer cells, the prostate cancer cells and the urinary bladder cancer cells to prove the cytotoxicity of flavonoid on the cancer cells and the signaling transduction pathway thereon. The clinical application value of the flavonoid compound is promoted.

In accordance with the first aspect of the present invention, a pharmaceutical composition for treating a cancer is provided. The pharmaceutical composition includes a flavonoid represented by formula I:

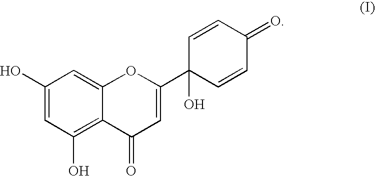

The cancer is one selected from a group consisting of a gynecological cancer, a prostate cancer, a bladder cancer and a liver cancer.

Preferably, the gynecological cancer is one selected from a group consisting of an ovarian cancer, a breast cancer and a cervical cancer. The ovarian cancer is demonstrated by one of an MDAH-2774 cell line and an cell line, the breast cancer is demonstrated by one of an MDA-MB-468 cell line, an MDA-C33A cell line and a T47D cell line, and the cervical cancer is demonstrated by a HeLa cell line.

Preferably, the prostate cancer is demonstrated by an LNCap cell line, the bladder cancer is demonstrated by one of an RT4 cell line and a T24 cell line, and the liver cancer is demonstrated by a Hep 3B cell line.

Preferably, the flavonoid is an extract of a fern which is Thelypteris torresiana (Gaud).

Preferably, the flavonoid arrests a cell at one of an S phase and a G2/M phase by regulating an expression of at least one cell cycle protein of the cell.

Preferably, the at least one cell cycle protein is one selected from a group consisting of a phosphated cyclin B1 (p-cyclin B1) protein, a cyclin B1 protein, a phosphated cyclin-dependent kinase 2 (p-Cdk2) protein, a Cdk2 protein and a cell division cycle 25 homolog C (Cdc25C) protein.

Preferably, the at lease one cell cycle protein is one selected from a group consisting of a caspase-3 protein, a poly(ADP)ribose polymerase (PARP) protein, a B-cell leukemia/lymphoma x long (Bcl-xL) protein and a B-cell leukemia/lymphoma 2 (Bcl-2) protein so as to induces the cell to go into an apoptosis.

Preferably, the flavonoid regulates one of a p38 mitogen-activated protein kinase (MAPK) protein, a c-Jun NH₂-terminal kinase (JNK) ½ protein and a combination thereof so as to induce the cell to go into an apoptosis.

Preferably, the pharmaceutical composition further includes cis-diamminedichloridoplatinum having a structure of Formula II:

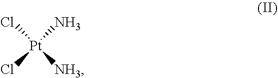

and synergistically functioning with the flavonoid to treat the cancer.

Preferably, the pharmaceutical composition further includes a pharmaceutically acceptable carrier.

Preferably, the flavonoid inhibits a cancer cell of a mammal which includes a human being and a rodent. The rodent includes a mouse and a rat.

In accordance with the second aspect of the present invention, a pharmaceutical composition for treating a cancer is provided. The pharmaceutical composition includes a flavonoid having a first structure of Formula I:

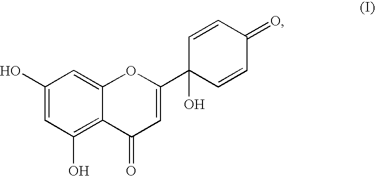

and a cis-diamminedichloridoplatinum having a second structure of Formula II:

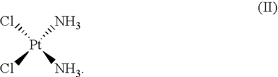

Preferably, the pharmaceutical composition inhibits a growth of a cancer cell.

The above objectives and advantages of the present invention will become more readily apparent to those ordinarily skilled in the art after reviewing the following detailed descriptions and accompanying drawings, in which:

### BRIEF DESCRIPTION OF THE DRAWINGS

FIG. 1 is the effect of protoapigenone on colony formation;

FIGS. 2(A) and 2(B) are the cell cycle distributions of the various concentrations of proptoapigenone treatment on ovarian cancer cells (A) MDAH-2774 and (B) SKOV3 for 24 hours;

FIG. 2(C) is the cell cycle distribution of the various concentrations of protoapigenone on SKOV3 cells for 4 hours;

FIG. 3 is the immunoblotting analysis of protein expression at S and G2/M phases regulated by protoapigenone;

FIG. 4(A) is the apoptosis of the ovarian cancer cells induced by 10 μM protoapigenone;

FIG. 4(B) is the sub-G1 phase of the ovarian cancer cells treated with 10 μM protoapigenone for 24 hours;

FIG. 5 is the protein expressions of the apoptotic cells induced by protoapigenone;

FIG. 6(A) is the cytotoxicity of MDAH-2774 cells treated with protoapigenone and cisplatin respectively or co-treated therewith;

FIG. 6(B) is the protein expression of MDAH-2774 cells treated with protoapigenone and cisplatin respectively or co-treated therewith for 24 hours;

FIG. 7 is the tumor growth on MDAH-2774-xenografted nude mice inhibited by protoapigenone;

FIG. 8 is the protein expression of the cleaved PARP in the ovarian tumor tissues;

FIG. 9 is the growth inhibition of various protoapigenone concentrations on the prostate cancer cell line LNCap;

FIG. 10(A) is the protoapigenone-induced apoptosis determined with annexin V-FITC assay;

FIG. 10(B) is the protoapigenone-induced apoptosis determined with TUNEL assay;

FIG. 10(C) is the protein expression of the cellular apoptosis induced by protoapigenone;

FIGS. 11(A) and 11(B) are the cell cycle distributions of LNCap cells treated with protoapigenone for (A) 6 hours and (B) 12 hours respectively;

FIG. 11(C) is the protein expressions of LNCap cells after the protoapigenone treatment for 6 and 12 hours;

FIGS. 12(A) and 12(B) are the protein expressions of LNCap cells treated with protoapigenone;

FIG. 13(A) is the protein expressions of LNCap cells treated with SB203580 or SP600125, then treated with protoapigenone for 1 hour;

FIG. 13(B) is the survival rate of LNCap cells treated with SB203580 and SP600125 respectively first, then treated with protoapigenone for 12 hours;

FIGS. 14(A) to 14(C), which are the effects of p38 MAPK inhibitor SB203580 and JNK½ inhibitor SP600125 on the protoapigenone-induced apoptosis and cell cycle arrest;

FIG. 15(A) is the levels of p38 MAPK and JNK½ attenuated by the p38 MAPK and JNK½ specific siRNAs;

FIG. 15(B) is the apoptotic percentages inhibited by p38 MPAK siRNA ans JNK½ siRNA;

FIG. 15(C) is the cell cycle distribution of the cellular apoptosis induced by protoapigenone inhibited by p38 MAPK and JNK½ siRNAs;

FIG. 15(D) is the protein expression induced by protoapigenone inhibited by p38 MAPK and JNK½ siRNAs;

FIG. 16(A) is the relationship between time and the tumor volume that the LNCap-xenografted rude mice are intraperitoneally injected with protoapigenone;

FIG. 16(B) is the protein expressions of the cleaved PARP, p-p38 MAPK and p-JNK½ in the prostate tumor of the LNCap-xenograft nude mice which are intraperitoneally injected with low- and high-dose protoapigenone;

FIGS. 17(A), 17(B) and 17(C) are growth inhibitions of (A) RT4, (B) T24 and (C) Hep 3B after various concentrations of protoapigenone treatment; and

FIG. 18 is the cell cycle distribution of protoapigenone on human cervical cancer cell line HeLa.

### DETAILED DESCRIPTION OF THE PREFERRED EMBODIMENT

The present invention will now be described more specifically with reference to the following Embodiments. It is to be noted that the following descriptions of preferred Embodiments of this invention are presented herein for purpose of illustration and description only; it is not intended to be exhaustive or to be limited to the precise form disclosed.

### Cellular Experiments

I. Experimental Materials:

Protoapigenone was isolated from the whole plant, Thelypteris torresiana (Gaud) (Lin et al., 2005), and was dissolved in dimethyl sulfoxide (DMSO) and added to the cultured cells at a 1 to 1000.

The gynecological cancer cell lines used in the present invention were listed as follows. (1) Human ovarian cancer cell lines MDAH-2774 and SKOV3, human breast cancer cell lines MDA-MB-468, MDA-MB-231 and T47D, human cervical cancer cell lines HeLa and C33A and the immortalized non-cancer human breast epithelial cell line MCF-10A were purchased from American Type Culture Collection (ATCC), Manassas, Va. These cell lines were incubated in DMEM-F12 medium supplemented with 10% of fetal bovine serum and penicillin/streptomycin/amphotericin B. (2) Immortalized human ovarian surface epithelial (HOSE) cell lines, HOSE 6-3 and HOSE 11-12, were provided by Professor George S. W. Tsao (University of Hong Kong, Poffulam, China). These cell lines were incubated in 1:1 mixture of MCDB 105 and M199 medium (Sigma, St. Louis, Mo.) supplemented with 10% of fetal calf serum and penicillin/streptomycin/amphotericin B.

Human prostate cancer cell line LNCap used in the present invention was purchased from ATCC, Manassas, Va. and was cultured in RPMI 1640 medium supplemented with 10% FBS and penicillin/streptomycin/amphotericin B. LNCap cells were treated with 2.5, 5 and 10 μM protoapigenone respectively and were analyzed. In some experiments, LNCap cells were treated with p38 mitogen-activated protein kinase (MAPK) inhibitor SB203580 or c-Jun NH₂-terminal kinase (JNK) ½ inhibitor SP600125 for one hour before protoapigenone treatment.

II. Experimental Methods:

1. XTT Cell Proliferation and Clonogenic Assay:

Cells were seeded at 7×10³ per well in 96-well culture plates before treatment with different concentrations of protoapigenone for 12, 24 and/or 48 hours. Next, the cytotoxicity of protoapigenone was determined using XTT cell proliferation assay (Sigma, St. Louis, Mo.). The absorbance of wavelength at 490 nm (OD₄₉₀) and 650 nm (OD₆₅₀) were determined respectively by enzyme-linked immunosorbent assay (ELISA) reader, and 50% of inhibitory concentration (IC₅₀) was calculated by the value of OD₄₉₀-OD₆₅₀. In addition, in order to evaluate the long-term effect, the cells were treated with different concentration of protoapigenone for 3 hours, then were incubated with fresh medium for 14 days so as to form colonies. Finally, the cells were stained with crystal violet and were observed under the microscope.

2. Cell Survival Assay:

SKOV3 cells at various cell cycle phases were incubated in the 6-cm Petri dishes and treated with protoapigenone at different dosages (0, 5 and 10 μM/ml). Subsequently, the cells were harvested and mixed with trypan blue at a ratio of 1:1. The stained apoptotic cells were observed under the microscope.

3. Cell Cycle and Sub-G1 Analysis:

The ovarian cancer cells and the prostate cancer cells respectively were treated with protoapigenone at different concentrations (0, 2.5, 5 and 10 μM) for 24 hours and 6 hours (or 12 hours). These cells were harvested by trypsin and fixed with 70% ethanol for 1 hour, washed with phosphate buffered saline (PBS) twice, and resuspended in propidium iodide (PI)/RNase A solution for 30 minutes. In accordance with the mechanism of DNA stained with propidium iodide, the cell cycle and sub-G1 distribution were detected by the FACScan flow cytometry (Becton Dickinson, San Jose, Calif.), and data analysis was done with CellQuest software (BD Bioscience).

4. Annexin V Apoptosis Assay:

The theory of this assay is to detect early apoptotic cells during the apoptotic progression. The ovarian cancer cells and the prostate cancer cells respectively were incubated with protoapigenone at 10 μM for 3 hours on chamber slides, washed with cold PBS twice and stained with a binding buffer containing annexin V-fluorescein isothiocyanate (FITC) (1 mg/ml; Strong Biotech, Taipei, Taiwan) and 4,6-diamidino-2-phenylinodole (Sigma-Aldrich) at 25° C. for 15 minutes. The stained cells were washed with PBS twice and observed under the fluorescent microscope.

5. Terminal Deoxynucleotidyl Transferase dUTP Nick-End Labeling (TUNEL) Assay:

TUNEL assay is performed to detect whether cellular DNA is fragmented. The prostate cancer cells were treated with protoapigenone at various concentrations (2.5, 5 and 10 μM) for 12 or 24 hours, then were stained with TUNEL using the DeadEnd Colorimetric TUNEL system (Promega, Madison, Wis.). In brief, the prostate cancer cells were fixed with 4% paraformaldehyde for 30 minutes after protoapigenone treatment. Next, the fixed cells were incubated with digoxigenin-conjugated dUTP and nucleotide mixture in a recombinant terminal deoxynucleotide transferase-catalyzed reaction in the humidified atmosphere at 37° C. Subsequently, they were immersed in the stop buffer for 15 minutes at 37° C. The Cells were washed with PBS, then were incubated in DAB solutions for 15 minutes in dark. The stained cells were observed under the microscope. The percentage of TUNEL-positive cells were counted in 1000 cells, and the apoptosis index in the later period was calculated.

6. Immunoblotting Analysis:

The cells treated with protoapigenone at various concentrations (0, 2.5, 5 and 10 μM) for 24 hours were washed twice and lysed with EBC buffer (50 mM Tris (pH 7.6), 120 mM NaCl, 0.5% Nonidet P-40, 1 mM β-mercaptoethanol, 50 mM NaF, and 1 mM Na₃VO₄). The concentration of protein supernatant was further determined using the Bio-Rad protein assay kit (Bio-Rad Laboratories, Hercules, Calif.). Whole cell lysates were separated on polyacrylamide sodium dodecyl sulfate (SDS) gels and the proteins were transferred onto the nitrocellulose membrane. Proteins were detected sequentially by the specific primary antibodies, followed by peroxidase-conjugated secondary antibodies, and visualized by the Enhanced chemiluminescence detection system (Amersham, N.J.).

7. Nude Mice Model:

MDAH-2774 human ovarian cancer cells of 2×10⁶ (or LNCap prostate cancer cells of 1×10⁶) in 0.1 ml PBS were injected subcutaneously at the right flank of 6-week-old female nude mice (Foxnlun/Foxulun). When tumors became visible (approximately 3×3 mm in size), the mice were randomly grouped and treated intraperitoneally with protoapigenone or vehicle every other day. In the xenograft experiment of MDAH-2774 human ovarian cancer cells, the control group, the low dose group and the high dose group were given PBS, 0.069 μM/g body weight of protoapigenone in PBS (a dose equals to one-tenth of the IC₅₀ for MDAH-2774 cells), and 0.69 μM/g body weight of protoapigenone in PBS (a dose equals to the IC₅₀ for MDAH-2774 cells) individually. In the xenograft experiment of LNCap prostate cancer cells, the control group, the low dose group and the high dose group were given PBS, 0.37 μM/g body weight of protoapigenone in PBS (a dose equals to one-tenth of the IC₅₀ for LNCap cells), and 3.7 μM/g body weight of protoapigenone in PBS (a dose equals to the IC₅₀ for LNCap cells) individually. Tumor size and body weight were measured twice a week using calipers, and their tumor volumes were calculated according to a standard formula: width²×length/2. After treatment with protoapigenone for 5 weeks, the mice xenografted with LNCap cells were sacrificed by deep anesthesia, and the blood sampling by cardiac aspiration was done immediately after anesthesia. Complete blood count of the nude mice blood was determined by Sysmex XE-2100 (TOA Medical Electronics, Kobe, Japan), and the plasma blood urea nitrogen (BUN), creatine (Cr), asparatate aminotransferase (AST), and alanine aminotransferase (ALT) levels were determined by Beckman LX20 (Beckman-Coulter, U.S.A.)

8. Small Interfering RNA (siRNA) Knockdown:

The theory of this assay is to attenuate the p38 MAPK and JNK½ expressions of LNCap cells using specific siRNA (Santa Cruz Biotechnology, Inc.) and transfecting with Lipofectamine 2000 (Invitrogen). LNCap cells were treated with protoapigenone after incubating for 48 hours, and the treated LNCap cells were harvested to analyze.

9. Immunohistochemical Analysis:

After the tissue specimen were fixed with 10% formaldehyde or 4% tripolyaldehyde (Yeh et al., 2006), the tissue samples were dissected, dehydrated and coated with wax. The thickness of specimen was 3 μm or 4 μm, and the specimen were immunostained with hematoxylin-eosin (H & E) or primary antibody, then were labeled with Universal LAB+kit/horseradish peroxidase (HRP) (Dako Denmark A/S, Glostrup, Denmark), or were counterstained with hematoxylin. The cells were observed under the microscope.

III. Experimental Results:

1. Inhibition of Protoapigenone on Ovarian Cancer Cells:

Please refer to Table 1, which is the cytotoxicity of protoapigenone on various cancer cell lines. In Table 1, protoapigenone has the highest cytotoxicity on the ovarian cancer cell lines MDAH-2774 and SKOV3; however, protoapigenone has the lower cytotoxicity on the immortalized human ovarian epithelial cells HOSE 6-3 and HOSE 11-12. The identical results can be corresponded to the breast cancer cells MDA-MB-468 and T47D and the immortalized human breast epithelial cells MCF-10A.

TABLE 1
Cytotoxicity of protoapigenone on various cancer cell lines

| | Cell line | IC₅₀ (μM) |
|---|---|---|
| Ovarian cancer | MDAH-2774 | 0.69 ± 0.92^{a} |
| | SKOV3 | 0.78 ± 0.28 |
| Cervical cancer | HeLa | 3.66 ± 0.61 |
| | C33A | 4.69 ± 0.21 |
| Breast cancer | MDA-MB-468 | 3.33 ± 1.25 |
| | T47D | 5.13 ± 0.23 |
| Immortalized human ovarian | HOSE 6-3 | 8.98 ± 0.33 |
| epithelial cells | HOSE 11-12 | 10.1 ± 0.53 |
| Immortalized human breast | MCF-10A | 33.6 ± 0.53 |
| epithelial cell | | |

^{a}Means ± SD of three independent experiments

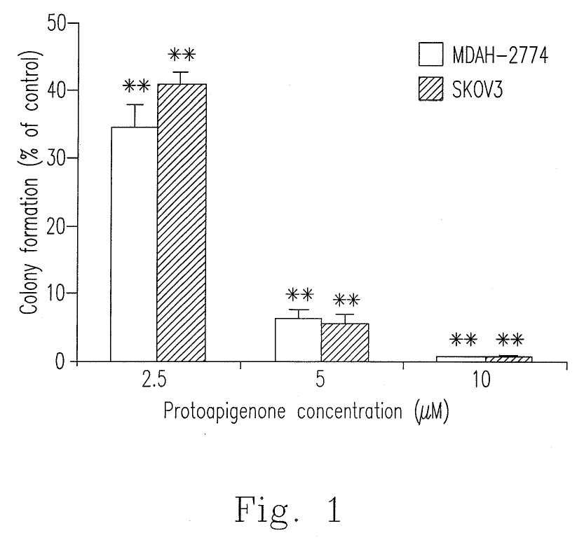

In addition, please refer to FIG. 1, which is the effect of protoapigenone on colony formation. In FIG. 1, the ratios of MDAH-2774 and colony formations decrease along with the increased protoapigenone concentrations. From Table 1 and FIG. 1, it is known that protoapigenone owns toxicity on the ovarian cancer cells and inhibits the growth thereof, but does not has toxicity on the immortalized ovarian epithelial cancer cells. It means that protoapigenone has selective cytotoxicity on ovarian cancer cells.

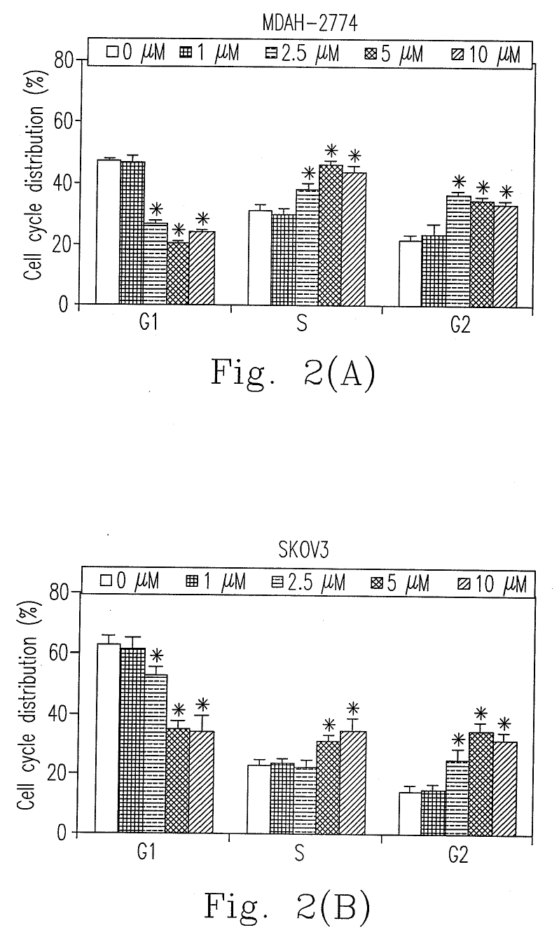

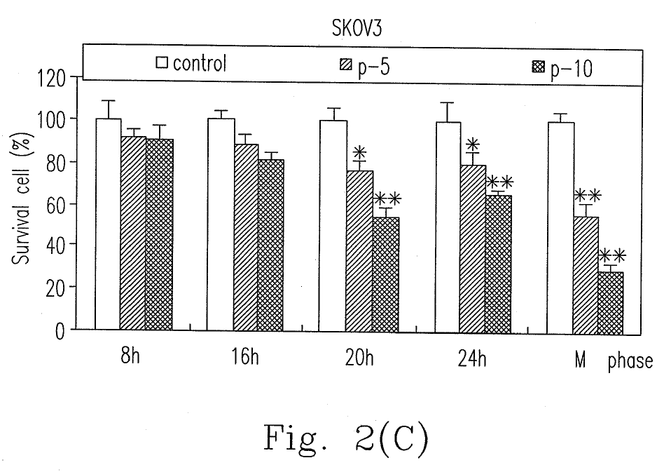

Please refer to FIGS. 2(A) and 2(B), which are the cell cycle distributions of the various concentrations of proptoapigenone treatment on ovarian cancer cells (A) MDAH-2774 and (B) SKOV3 for 24 hours. In FIG. 2(A) and 2(B), protoapigenone significantly makes the MDAH-2774 and SKOV3 cells enter into S and G2/M phases, and the cells entering into S and G2/M phases are increased along with the increased protoapigenone concentrations. Please refer to FIG. 2(C), which is the cell cycle distribution of the various concentrations of protoapigenone on SKOV3 cells for 4 hours. In FIG. 2(C), the number of SKOV3 cells proliferated at S, G2 and M phases decreases 23.5% and 45.4%, 20.3% and 34.7%, and 44.8% and 70.5% respectively while SKOV3 cells are treated with 5 μM (i.e. p-5) and 10 μM (i.e. p-10) protoapigenone.

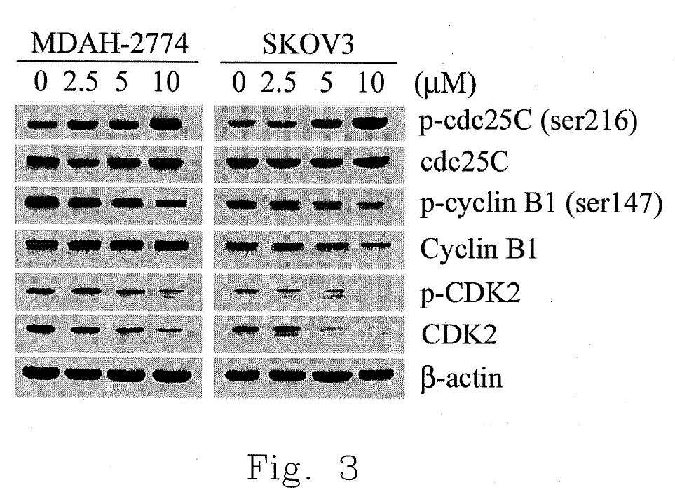

Please refer to FIG. 3, which is the immunoblotting analysis of protein expression at S and G2/M phases regulated by protoapigenone. In FIG. 3, the protein expressions of phosphated cyclin-dependent kinase 2 (p-Cdk2), Cdk2, phosphated cyclin B1 (p-Cyclin B1) Ser¹⁴⁷ and Cyclin B1 in MDAH-2774 and SKOV3 cells decrease but the expression of p-Cdc25C (Ser²¹⁶) along with the increased protoapigenone concentrations. Since Cdk2, Cyclin B1 and Cdc25C belong to the proteins for inhibiting and regulating the cell cycle, the phosphorylated/inactivated Cdc25C (Ser²¹⁶) binds to the member of 14-3-3 family and sequesters in the cytoplasm and therefore prevents the premature mitosis (Hirose et al., 2001). The phosphorylation of Cyclin B1 is required for entry into M phase in a Cdc25C-dependent manner (Toyoshima-Morimoto et al., 2002). From FIG. 3, it is known that protoapigenone increases the phosphorylation of Cdc25C (Ser²¹⁶) and decreases the expressions of Cdk2 (Thr¹⁶¹) and Cyclin B1 (Ser¹⁴⁷). Therefore, from FIGS. 2(C) and 3, it is known that protoapigenone has significant cytotoxicity on SKOV3 cells at S and G2/M phases. Protoapigenone not only arrests SKOV3 cells at S and G2/M phases but also causes the most significant cytotoxicity on SKOV3 cells at these phases. Protoapigenone indeed arrests the cell cycles of the ovarian cancer cells by regulating the cell cycle protein expressions thereof.

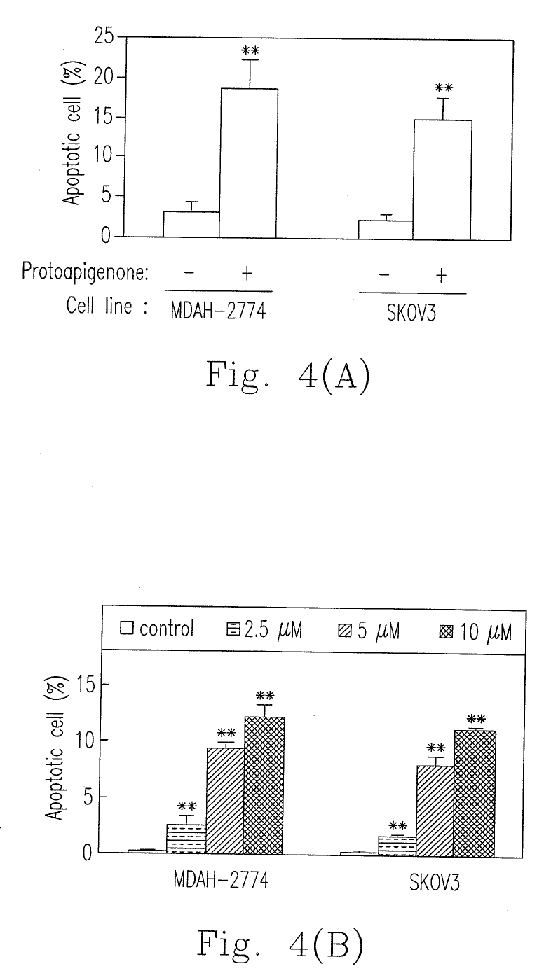

A quantitative evaluation of apoptosis is sought using an annexin V-FITC dye to detect the translocation of phosphatidylserine from cytoplasmic leaflet of the plasma membrane to the cell surface. Please refer to FIG. 4(A), which is the apoptosis of the ovarian cancer cells induced by 10 μM protoapigenone. In FIG. 4(A), comparing with the control, 10 μM protoapigenone treatment for 3 hours induces 24.5% and 34.2% of cell apoptosis in MDAH-2774 and SKOV3 cells respectively. Please refer to FIG. 4(B), which is the sub-G1 phase of the ovarian cancer cells treated with 10 μM protoapigenone for 24 hours. In FIG. 4(B), the protoapigenone treatment increases the apoptotic sub-G1 peak of MDAH-2774 and SKOV3 cells in a dose-dependent manner.

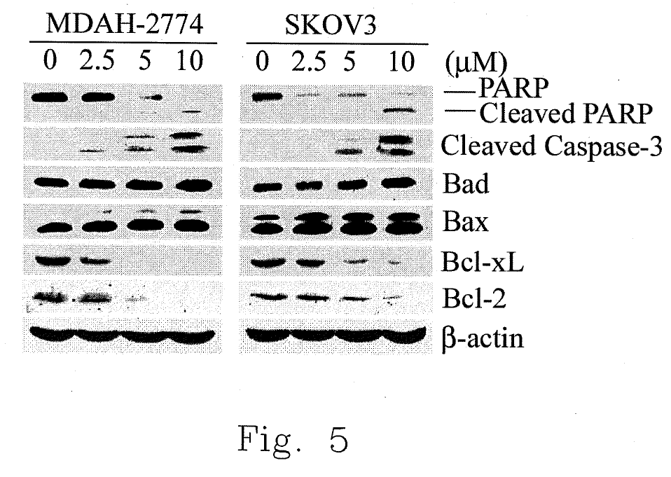

Please refer to FIG. 5, which is the protein expressions of the apoptotic cells induced by protoapigenone. Since the cleaved poly(ADP)ribose polymerase (PARP) is an apoptotic marker, and is activated by caspase-3 which is further regulated by Bcl-2 protein family, protoapigenone decreases expressions of Bcl-2 and Bcl-xL on the ovarian cancer cells, activates caspase-3 and cleaves the intact PARP to induce cells to enter into apoptosis. In addition, protoapigenone can increases the expressions of Bcl-2-associated death promoter (Bad) and Bcl-2-associated X (Bax) proteins.

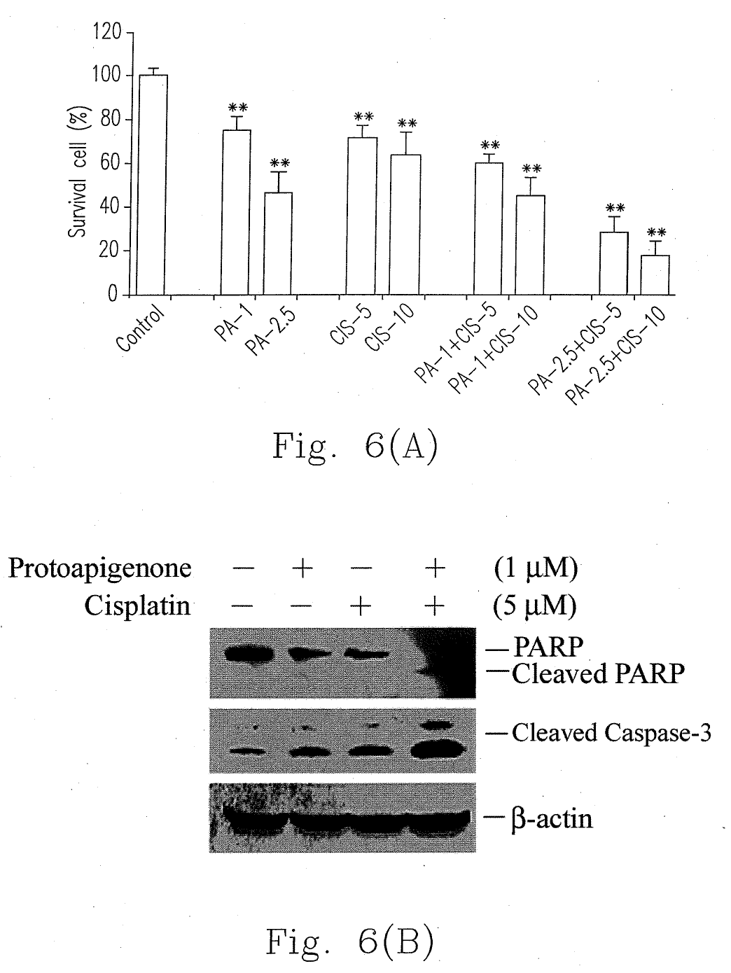

In the past clinical research, cisplatin (also named as cisplatinum or cis-diamminedichloridoplatinum (II), CDDP, as Formula II) is a common, platinum-based anti-cancer or chemotherapeutic agent for treating cancers, such as sarcoma, small cell lung cancer, ovarian cancer, lymphoma and germ cell tumor, etc. Cisplatin forms platinum complex bounded with or interconnected with DNA in the cells, and finally triggers apoptosis program or automatic apoptosis. Therefore, a pharmaceutical composition including protoapigenone and cisplatin is evaluated whether the pharmaceutical composition has synergistic cytotoxicity on cells. Please refer to FIG. 6(A), which is the cytotoxicity of MDAH-2774 cells treated with protoapigenone and cisplatin respectively or co-treated therewith. In FIG. 6(A), 1 and 2.5 μM protoapigenone (abbreviated as PA in FIG. 6(A)) treatments inhibit 24.96% and 53.36% of cell growth respectively while 5 and 10 μM cisplatin (abbreviated as CIS in FIG. 6(A)) treatments inhibit 28.48% and 36.91% of cell growth respectively. The combination treatment of 1 μM protoapigenone with 5 or 10 μM cisplatin inhibits 39.73% and 55.40% of cell growth, while the combination treatment of 2.5 μM protoapigenone with 5 or 10 μM cisplatin inhibits 72.02% and 82.65% of cell growth. Therefore, protoapigenone not only inhibits the growth of ovarian cancer cell individually but also reveals higher cytotoxicity effect co-treated with cisplatin.

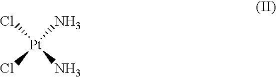

Please refer to FIG. 6(B), which is the protein expression of MDAH-2774 cells treated with protoapigenone and cisplatin respectively or co-treated therewith for 24 hours. In FIG. 6(B), comparing with the control (i.e. the group without protoapigenone and cisplatin treatment) or with the groups treating with protoapigenone and cisplatin individually, the cells in 1 μM protoapigenone and 5 μM cisplatin treatment can induce the intact PARP cleaved and activate the activity of caspase-3.

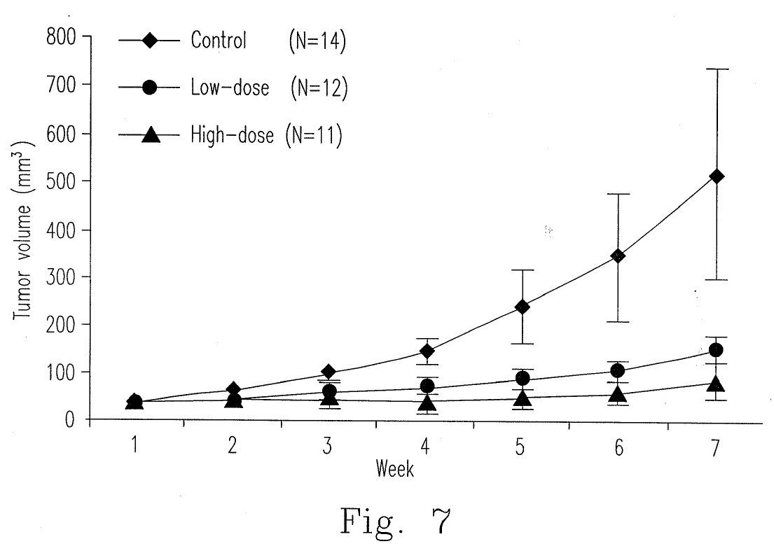

Please refer to FIG. 7, which is the tumor growth on MDAH-2774-xenografted nude mice inhibited by protoapigenone. In FIG. 7, comparing with the control (PBS treatment), the tumor growth of MDAH-2774 cells is significantly suppressed by protoapigenone at the low dose and high dose after 7-week treatment. Please refer to Table 2, which is (A) the Body weight profile, (B) complete blood count and biochemical profile for the MDAH-2774-xenografted nude mice after treatment with protoapigenone for 7 weeks. In Table 2, it is found that no significant impairment of hematopoiesis, liver function or renal function is observed in the nude mice after intraperitoneal injection of protoapigenone.

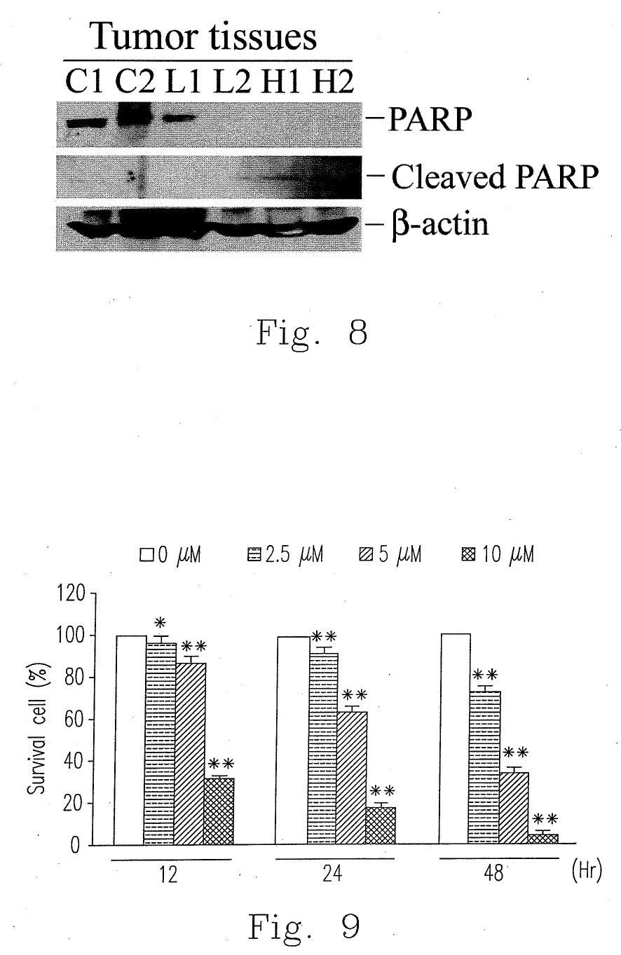

The MDAH-2774 tumor xenografted from the control and the protoapigenone-treatment mice are harvested, and apoptosis is accessed by immunoblotting and immunohistochemical analysis. Please refer to FIG. 8, which is the protein expression of the cleaved PARP in the ovarian tumor tissues. In FIG. 8, the cleave PARP is increased in the protoapigenone-treated tumor tissues but not in the control. In addition, protoapigenone induces the apoptosis of the MDAH-2774 xenografts, and the nuclear expression of the cleaved PARP is significantly increased in the protoapigenone-treated tumor tissues.

2. Inhibition of Prostate Cancer Cells by Protoapigenone:

Please refer to FIG. 9, which is the growth inhibition of various protoapigenone concentrations on the prostate cancer cell line LNCap. In FIG. 9, the inhibition of protoapigenone on LNCap cells is increased along with time and the increased protoapigenone concentrations. The 48-hour protoapigenone treatment can obtain the highest inhibition effect with an IC₅₀ value of 3.7±0.2 μM; however, the cells in the 12-hour and 24-hour protoapigenone treatment represent shrinked (data not shown).

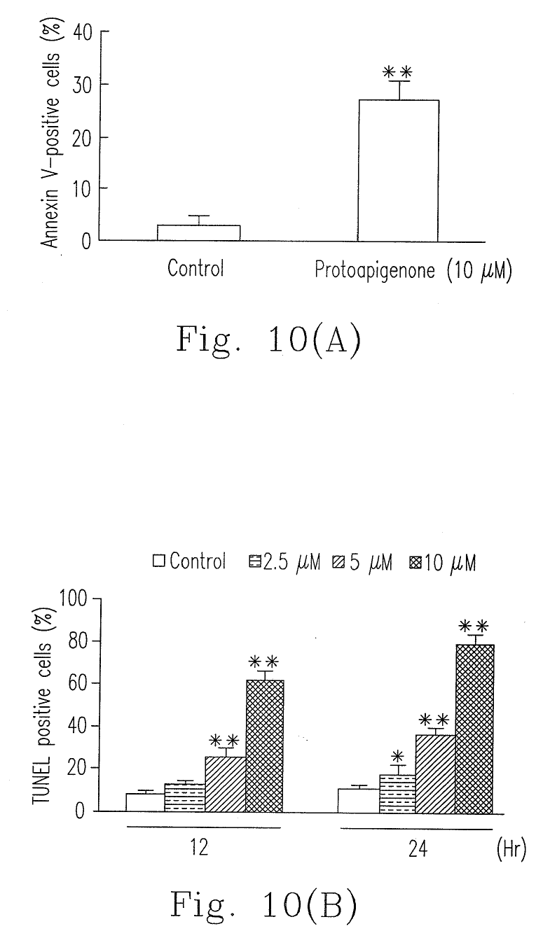

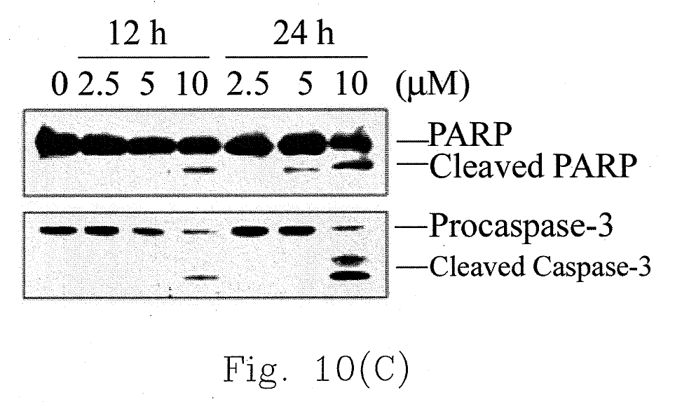

Please refer to FIG. 10(A), which is the protoapigenone-induced apoptosis determined with annexin V-FITC assay. In FIG. 10(A), after LNCap cells are treated with 10 μM protoapigenone for 3 hours, the ratio of the annexin V-FITC positive cells is increased 24.2±1.3%, but that of the control is only increased 2.7±1.0%. Please refer to FIG. 10(B), which is the protoapigenone-induced apoptosis determined with TUNEL assay. In FIG. 10(B), the ratio of the TUNEL positive cells are increased along with time and the increased protoapigenone concentrations. Please refer to FIG. 10(C), which is the protein expression of the cellular apoptosis induced by protoapigenone. In FIG. 10(C), the expressions of the cleaved PARP and the cleaved caspase-3 is increased along with time and the increased protoapigenone concentrations.

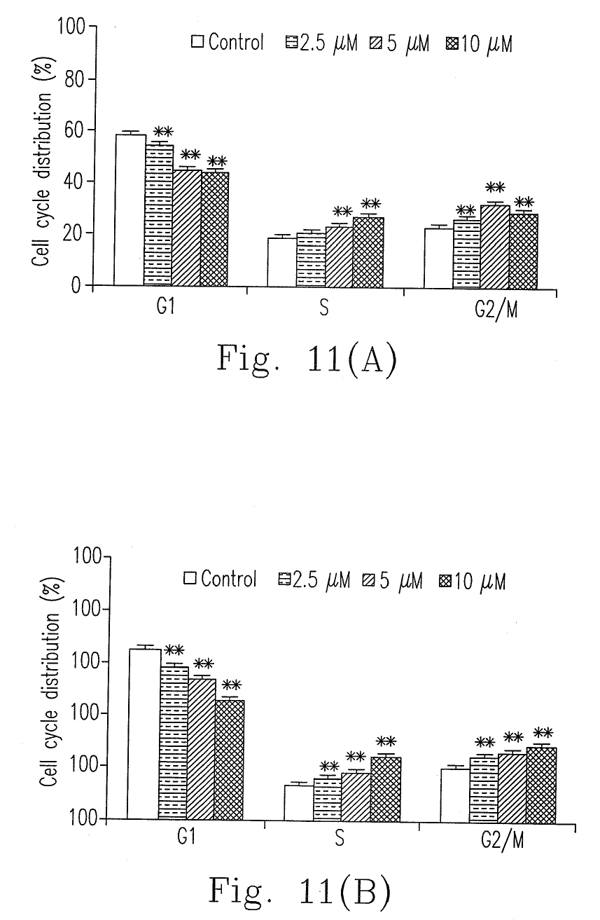

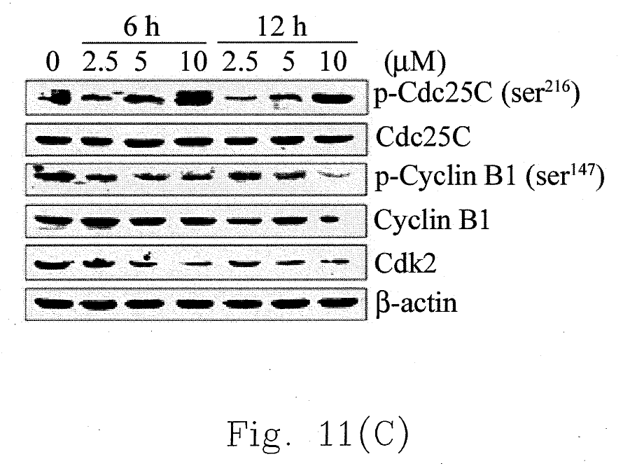

Please refer to FIGS. 11(A) and 11(B), which are the cell cycle distributions of LNCap cells treated with protoapigenone for (A) 6 hours and (B) 12 hours respectively. In FIGS. 11(A) and 11(B), protoapigenone arrests the LNCap cells at S and G2/M phases, and inhibits the progress of cell cycle. Please refer to FIG. 11(C), which is the protein expressions of LNCap cells after the protoapigenone treatment for 6 and 12 hours. In FIG. 11(C), the level of Cdk2 is increased along with time and the increased protoapigenone concentrations. After the 6-hour protoapigenone treatment, the activated p-cyclin B1 (Ser¹⁴⁷) is decreased; however, the activated p-cyclin B1 (Ser¹⁴⁷) and cyclin B1 all are decreased after the 12-hour protoapigenone treatment. The inactivated p-Cdc25C (Ser²¹⁶) all are increased after the 6-hour and 12-hour protoapigenone treatment respectively.

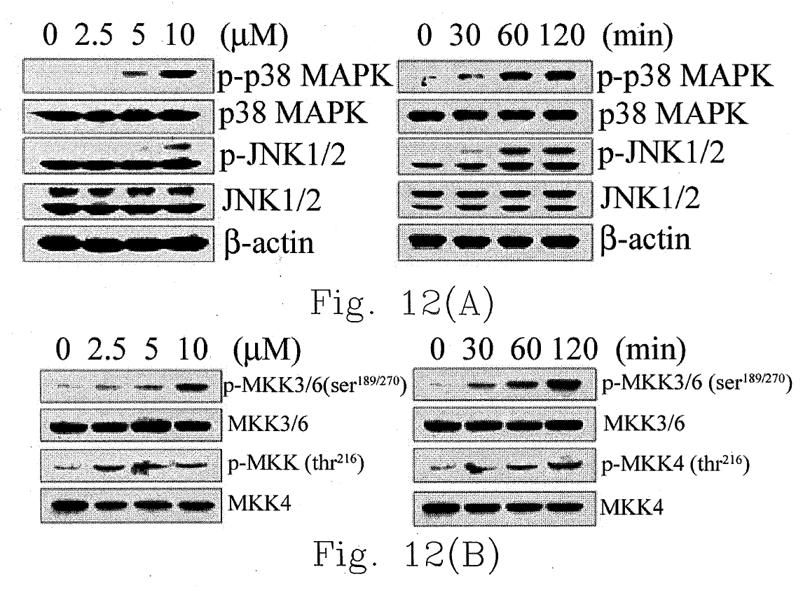

The anticancer activity of several flavonoids, including (−)-epigallocatechin gallate (a catechin, abbreviated as EGCG) and apigenin, are associated with the modulation of MAPK pathway (Kim et al., 2005; Van Dross et al., 2003). Thus, whether the MAPK pathway-related proteins of LNCap cells after the protoapigenone treatment are involved is further determined. Please refer to FIGS. 12(A) and 12(B), which are the protein expressions of LNCap cells treated with protoapigenone. In FIGS. 12(A) and 12(B), exposure of LNCap cells to protoapigenone for 1 hour results in a dramatic increase in the phosphorylation of p38 MAPK and JNK½. The phosphorylation of MAPK kinase (MKK) 3/6 (p-MKK 3/6 (Ser^{189/270})) and MKK4 (p-MKK4 (Thr²¹⁶)), the upstream kinases of p38 MAPK and JNK½, are also increased. Alternatively, the expression of the total forms of MKK 3/6, MKK4, p38 MAPK and JNK½ is not altered by the protoapigenone treatment, neither is the extracellular signal-regulated kinase (ERK) phosphorylation (data not shown).

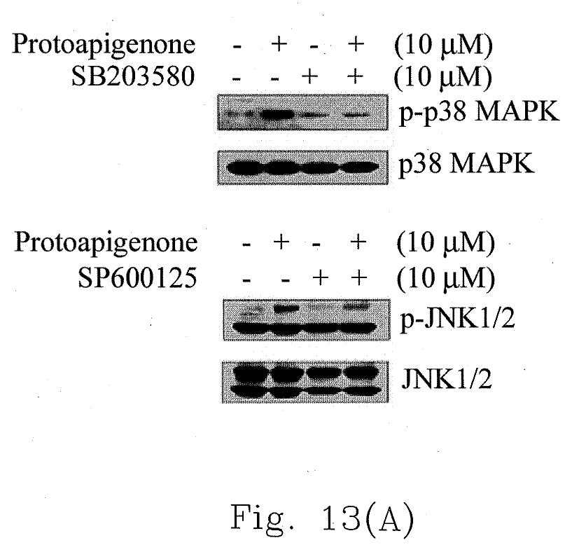

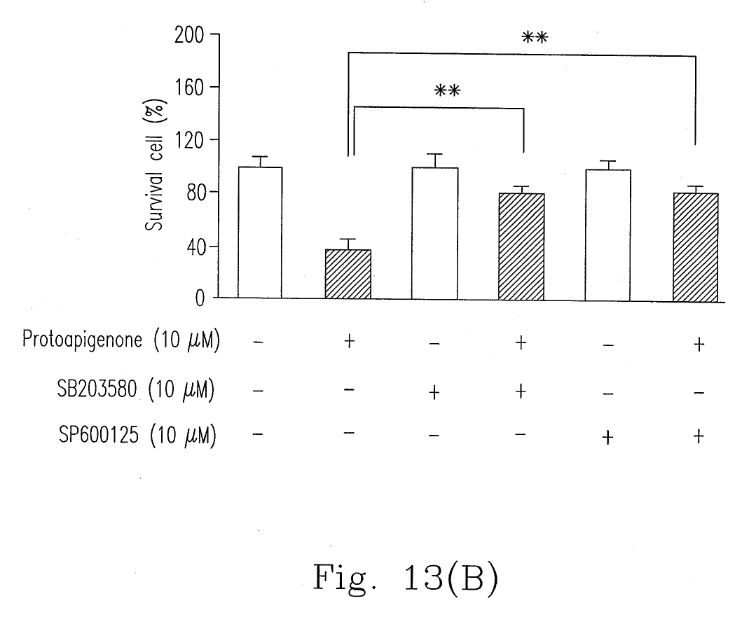

To evaluate the role of p38 MAPK and JNK½ activation in the protoapigenone-induced cell death, LNCap cells are pretreated for 1 hour with the pharmacological inhibitors of p38 MAPK (SB203580) and JNK½ (SP600125) respectively (FIG. 13(A)). The protoapigenone-induced phosphorylation (activation) of p38 MAPk and JNK½ is blocked in the presence of SB203580 and SP600125, respectively. Please refer to FIG. 13(B), which is the survival rate of LNCap cells treated with SB203580 and SP600125 respectively first, then treated with protoapigenone for 12 hours. In FIG. 13(B), the survival rates of the LNCap cells in the SB203580 and SP600125 treatments respectively, then the protoapigenone treatment all are higher than that of the LNCap cells only treated with protoapigenone. It means that the inactivation of p38 MAPK and JNK½ induces the protoapigenone-inhibited LNCap cells to proliferate.

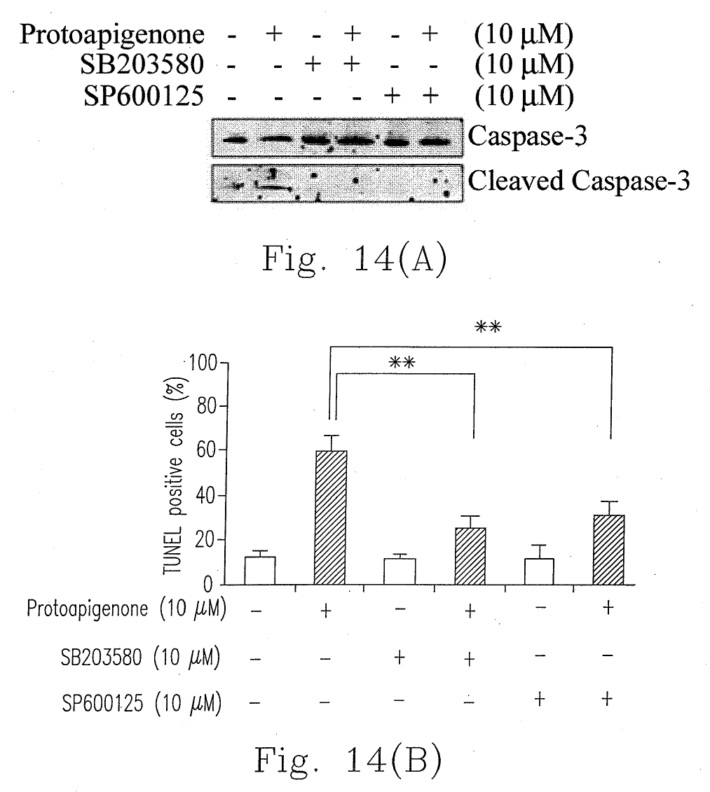

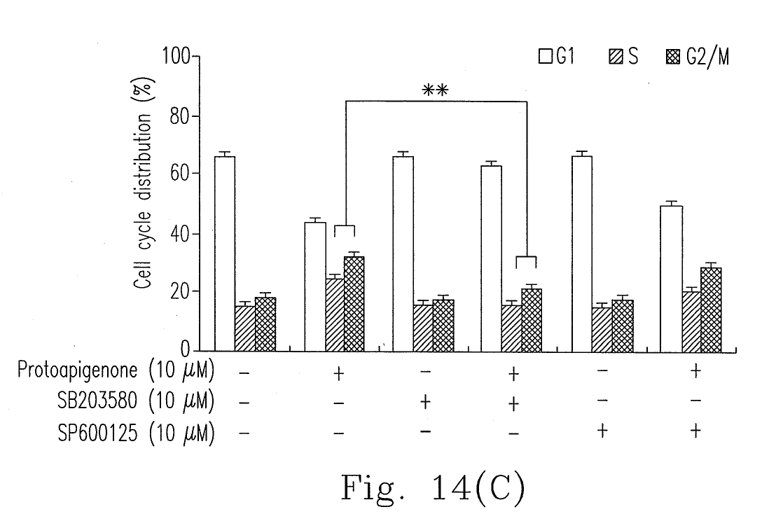

Whether the activation of p38 MAPK and JNK½ is involved in the protoapigenone-induced apoptosis and cell cycle arrest is further investigated. Please refer to FIGS. 14(A) to 14(C), which are the effects of p38 MAPK inhibitor SB203580 and JNK½ inhibitor SP600125 on the protoapigenone-induced apoptosis and cell cycle arrest. In FIGS. 14(A) and 14(B), the protoapigenone-induced apoptotic percentage is decreased by SB203580 and SP600125. In addition, the levels of the cleaved caspase-3 are abrogated in the presence of SB203580 and SP600125. In FIG. 14(C), SB203580, but not SP600125, significantly attenuates the protoapigenone-induced S and G2/M phases arrest. The level of p-Cdc25C (Ser²¹⁶) is decreased and that of Cdk2 are increased in the presence of SB203580. The inhibitor SP600125 has to significant effect on the protoapigenone-induced cell cycle arrest (data not shown).

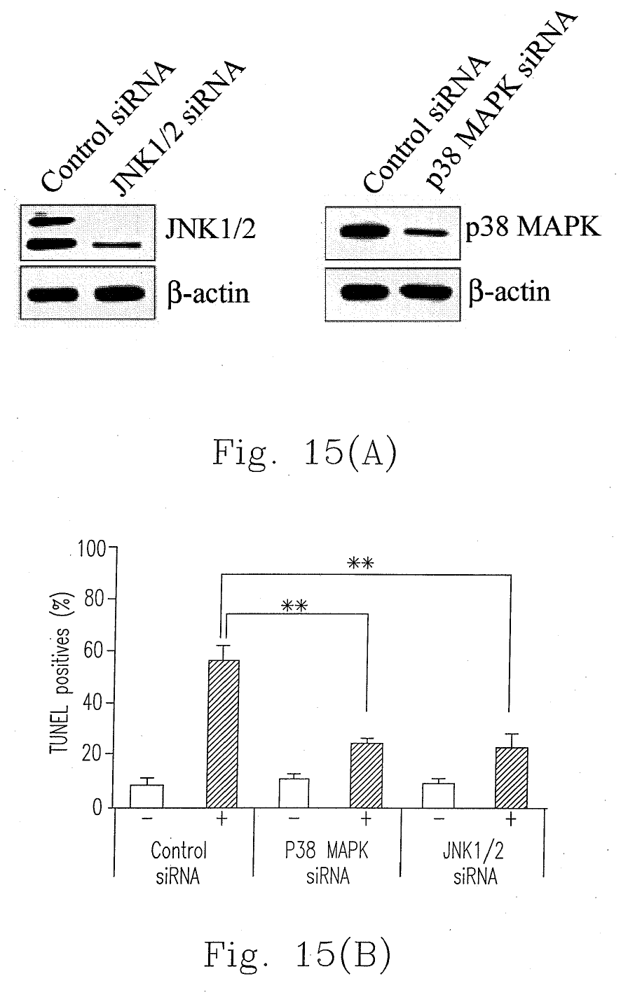

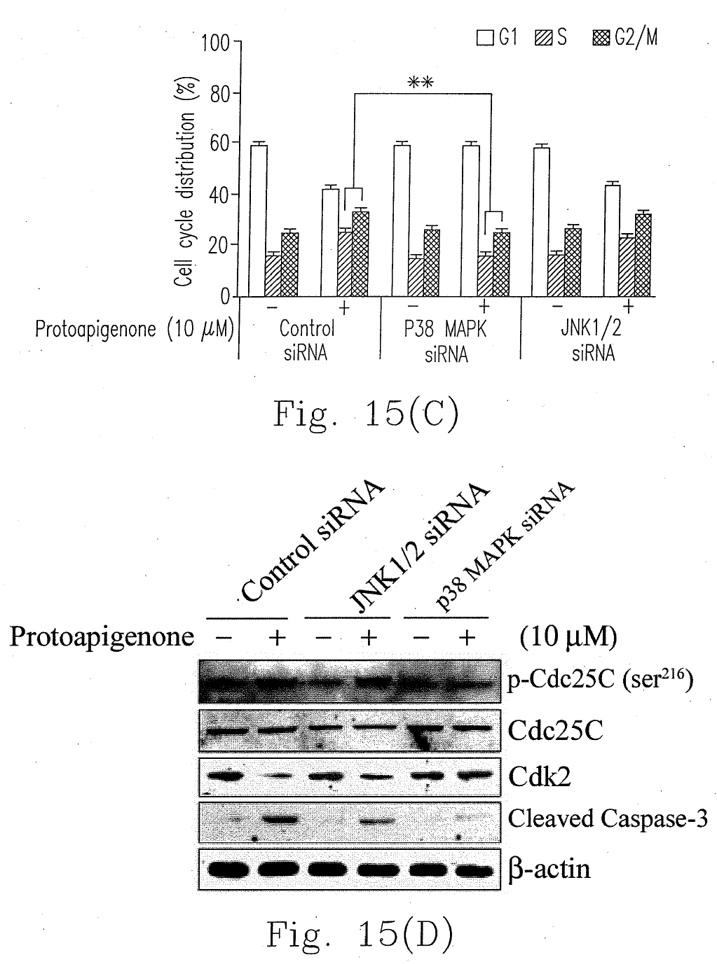

To further investigate the role of p38 MAPK and JNK½ in the protoapigenone-induced cell death, the siRNA specific for p38 MAPK and JNK½ are applied to knockdown the expression of p38 MAPk and JNK½, respectively (FIG. 15(A)). Further, the protoapigenone-induced apoptosis can be arrested by p38 MAPK siRNA and JNK½ siRNA (FIG. 15(B)). Please refer to FIG. 15(C), the selective inhibition of p38 MAPK, but not JNK½, releases the protoapigenone-induced S and G2/M phase arrest by modulating the levels of Cdk2 and the inactivated p-Cdc25C (Ser²¹⁶) (FIG. 15(D)). All these results are consistent with the results in FIGS. 14(A) to 14(C).

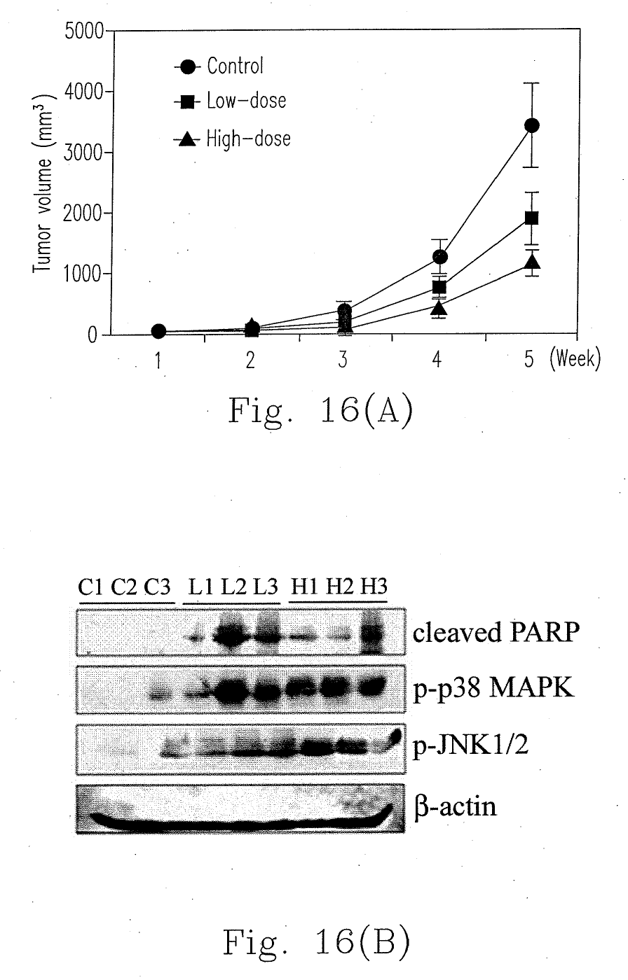

To determine whether protoapigenone suppresses the prostate cancer cell growth in vivo, LNCap cells are injected into the right flank of the rude mice, and the inhibition of tumor growth by protoapigenone is analyzed. Please refer to FIG. 16(A), which is the relationship between time and the tumor volume that the LNCap-xenografted rude mice are intraperitoneally injected with protoapigenone. In FIG. 16(A), after the protoapigenone treatment for 5 weeks, the average tumor size in high-dose (1153.0±218.7 mm³) and low-dose (1876.9±428.6 mm³) is significantly smaller than that in the control group (3409.8×704.8 mm³). No significant impairment of hematopoiesis, liver function, or renal function is observed after the intraperitoneal injection of protoapigenone, and the body weight has no significant difference between the control and the protoapigenone-treated group (Table 3). In addition, the levels of the cleaved PARP as well as the phosphorylation of p38 MAPK and JNK½ are increased in the protoapigenone-treated tumor tissues compared with the control (FIG. 16(B)). The unphosphorylated forms of p38 MAPK and JNK½ are not altered by the protoapigenone treatment (data not shown).

From the above-mentioned results, it is known that the activation of p38 MAPK and JNK½ signaling pathways, the increased levels of the cleaved PARP and caspase-3 and apoptosis induction in LNCap prostate cancer cells can be proceeded by protoapigenone treatment. The activities of p38 MAPK and JNK½ respectively can be inhibited by its inhibitor SB203580 and SP600125 and by the specific siRNA so as to decrease apoptosis, and the activities of the cleaved caspase-3 is proved by the present invention. Therefore, the important role of p38 MAPK and JNK½ on the protoapigenone-induced apoptosis is proved in the present invention.

In the present invention, the activated p38 MAPK and JNK½ induce the cell cycle to be arrested in G1/S or G2/M phase, which is achieved by increasing the inactivated p-Cdc25C (Ser²¹⁶) and by decreasing the levels of p-cyclin B1 (Ser¹⁴⁷) and Cdk2.

3. Inhibition of Other Cancer Cells by Protoapigenone:

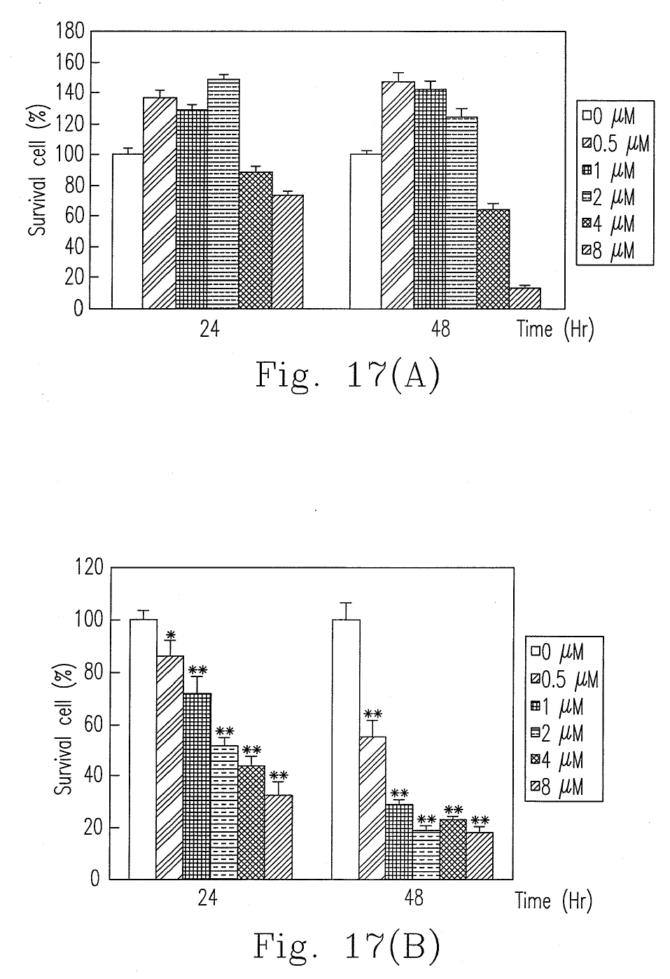

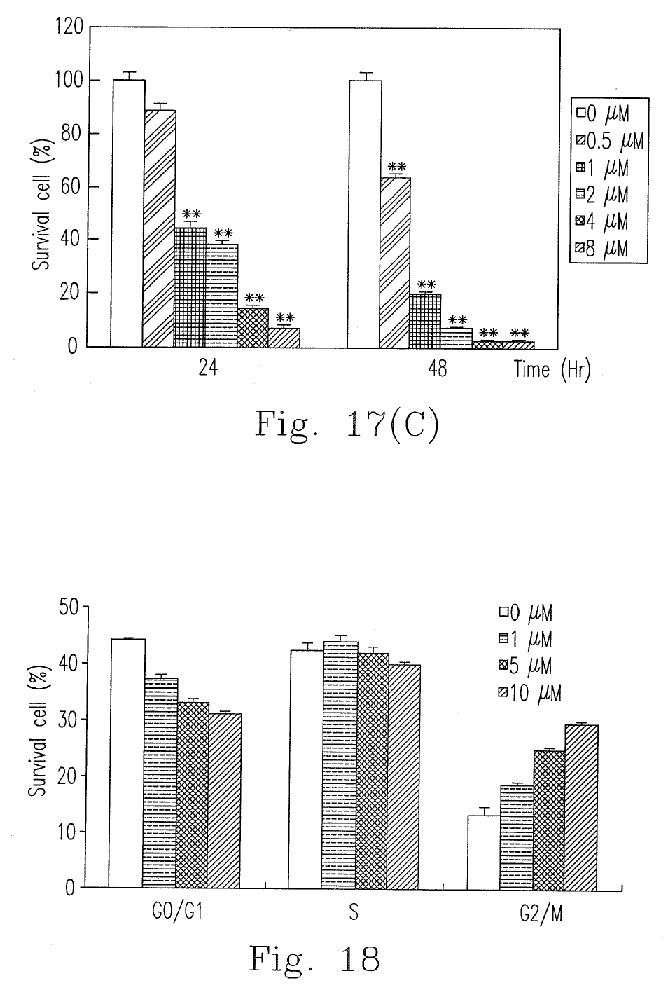

In addition to ovarian cancer and prostate cancer, bladder cancer, liver cancer and cervical cancer are also the research targets of the present invention to investigate whether the growth of these cancer cells can be inhibited by protoapigenone. First of all, the IC₅₀ values of protoapigenone on human urinary bladder transitional cell papilloma RT4 (ATCC number: HTB-2), T24 (ATCC number: HTB-4) and human hepatocellular carcinoma Hep 3B (ATCC number: HB-8064) respectively are 5.105±3.25 μM, 5.52±0.598 μM and 1.86=0.656 μM. Please refer to FIGS. 17(A), 17(B) and 17(C), which are growth inhibitions of (A) RT4, (B) T24 and (C) Hep 3B after various concentrations of protoapigenone treatment. In FIG. 17(A), the survival rate of RT4 cells are decreased by 4 μM- and 8 μM-protoapigenone treatment for 24 and 48 hours. However, the survival rate at low concentrations (0.5, 1 and 2 μM) of protoapigenone is over 100%, it is supposed that protoapigenone at low concentration would stimulate the cellular growth. In FIGS. 17(B) and 17(C), the growth inhibition of protoapigenone on T24 and Hep 3B cells is in a dose-dependent and time-dependent manners.

Please refer to FIG. 18, which is the cell cycle distribution of protoapigenone on human cervical cancer cell line HeLa. In FIG. 18, HeLa cells are arrested at G2/M phase by protoapigenone, and the mitosis of HeLa cells are arrested and is progressed into apoptosis.

In conclusion, apoptosis of the ovarian cancer cells, the prostate cancer cells, the urinary bladder cancer cells and the cervical cancer cells can be induced effectively by the protoapigenone treatment, and the signaling pathways thereof are proved. No significant hepatotoxicity, nephrotoxicity and hematological toxicity of protoapigenone on the nude mice renders that protoapigenone can be the chemotherapeutic agent on human and other mammals. Further, protoapigenone of the present invention and cisplatin can be combined as the pharmaceutical composition to achieve the effective effect of cancer therapy. Protoapigenone of the present invention can be manufactured as the dosage form with the pharmaceutically acceptable carrier.

While the invention has been described in terms of what is presently considered to be the most practical and preferred Embodiments, it is to be understood that the invention needs not be limited to the disclosed Embodiments. On the contrary, it is intended to cover various modifications and similar arrangements included within the spirit and scope of the appended claims, which are to be accorded with the broadest interpretation so as to encompass all such modifications and similar structures.

TABLE 2
(A) Body weight profile and (B) complete blood count and biochemical profile for the MDAH-2774-
xenografted nude mice after treatment with protoapigenone for 7 weeks
(A)

| | Control (N = 14) | | Low dose (N = 12) | | High dose (N = 11) | |
|---|---|---|---|---|---|---|
| Body weight (g) | Before treatment | After treatment | Before treatment | After treatment | Before treatment | After treatment |
| Means ± SD | 19.4 ± 0.3 | 19.8 ± 0.1 | 19.6 ± 0.6 | 19.4 ± 0.1 | 19.6 ± 0.8 | 19.8 ± 1.1 |

(B)

| Means ± SD | Control (N = 14) | Low dose (N = 12) | High dose (N = 11) |
|---|---|---|---|
| Got (U/L)^{a} | 135.6 ± 52.2 | 119.5 ± 45.5 | 148.2 ± 59.3 |
| Gpt (U/L)^{b} | 44.0 ± 23.4 | 46.3 ± 16.7 | 52.0 ± 17.6 |
| WBC (10 × 3/μl) | 3.0 ± 3.9 | 2.6 ± 1.2 | 2.0 ± 0.9 |
| RBC (10 × 3/μl) | 8.8 ± 0.3 | 7.8 ± 1.4 | 9.1 ± 0.3 |
| HCT (%)^{c} | 45.8 ± 2.0 | 43.5 ± 3.6 | 46.1 ± 1.8 |
| MCV (fl)^{d} | 51.7 ± 1.2 | 56.2 ± 8.9 | 50.9 ± 0.9 |
| PLT (10 × 3/μl) | 929.2 ± 142.7 | 1094.5 ± 124.9 | 916.3 ± 100.5 |
| Creatine(mg/dl) | 0.5 ± 0.0 | 0.5 ± 0.0 | 0.5 ± 0.0 |

^{a}Got means glutamic-oxaloacetic transaminase
^{b}Gpt means glutamic-pyruvic transaminase
^{c}HCT means hematocrit
^{d}MCV means mean corpuscular volume

TABLE 3
(A) Body weight profile and (B) complete blood count and biochemical profile for the LNCap-
xenografted nude mice after treatment with protoapigenone for 5 weeks
(A)

| | Control (N = 13) | | Low dose (N = 13) | | High dose (N = 11) | |
|---|---|---|---|---|---|---|
| Body weight (g) | Before treatment | After treatment | Before treatment | After treatment | Before treatment | After treatment |
| Means ± SD | 11.9 ± 0.591 | 14.0 ± 0.5 | 11.8 ± 0.4 | 13.6 ± 1.2 | 11.5 ± 0.8 | 13.5 ± 0.6 |

(B)

| Means ± SD | Control (N = 14) | Low dose (N = 12) | High dose (N = 11) |
|---|---|---|---|
| Got (U/L)^{a} | 581.1 ± 151.5 | 520.5 ± 169.9 | 630.7 ± 165.5 |
| Gpt (U/L)^{b} | 87.2 ± 53.5 | 130.0 ± 119.9 | 137.3 ± 55.8 |
| WBC (10 × 3/μl) | 3.9 ± 2.8 | 2.3 ± 0.7 | 2.7 ± 1.0 |
| RBC (10 × 3/μl) | 7.8 ± 0.6 | 7.3 ± 0.6 | 7.3 ± 0.7 |
| MCV (fl)^{c} | 46.4 ± 0.6 | 46.8 ± 0.8 | 47.7 ± 0.9 |
| BUN (mg/dl) | 25.5 ± 4.1 | 23.9 ± 3.2 | 24.8 ± 9.0 |
| Creatine (mg/dl) | 0.43 ± 0.06 | 0.43 ± 0.05 | 0.48 ± 0.10 |

^{a}Got means glutamic-oxaloacetic transaminase
^{b}Gpt means glutamic-pyruvic transaminase
^{c}MCV means mean corpuscular volume

What is claimed is:

1. A pharmaceutical composition for treating a cancer, comprising a flavonoid represented by formula I: 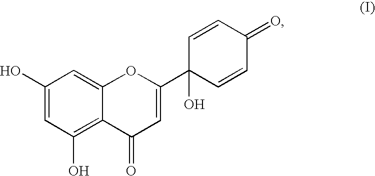
wherein the cancer is one selected from a group consisting of a gynecological cancer, a prostate cancer, a bladder cancer and a liver cancer.

2. The pharmaceutical composition according to claim 1, wherein the gynecological cancer is one selected from a group consisting of an ovarian cancer, a breast cancer and a cervical cancer.

3. The pharmaceutical composition according to claim 2, wherein the ovarian cancer is demonstrated by one of an MDAH-2774 cell line and an SKOV3 cell line, the breast cancer is demonstrated by one of an MDA-MB-468 cell line, an MDA-C33A cell line and a T47D cell line, and the cervical cancer is demonstrated by a Hela cell line.

4. The pharmaceutical composition according to claim 1, wherein the prostate cancer is demonstrated by an LNCap cell line.

5. The pharmaceutical composition according to claim 1, wherein the bladder cancer is demonstrated by one of an RT4 cell line and a T24 cell line.

6. The pharmaceutical composition according to claim 1, wherein the liver cancer is demonstrated by a Hep 3B cell line.

7. The pharmaceutical composition according to claim 1, wherein the flavonoid is an extract of a fern.

8. The pharmaceutical composition according to claim 7, wherein the fern is a Thelypteris torresiana.

9. The pharmaceutical composition according to claim 1, wherein the flavonoid arrests a cell at one of an S phase and a G2/M phase by regulating an expression of at least one cell cycle protein of the cell.

10. The pharmaceutical composition according to claim 9, wherein the at least one cell cycle protein is one selected from a group consisting of a phosphated cyclin B1 (p-cyclin B1) protein, a cyclin B1 protein, a phosphated cyclin-dependent kinase 2 (p-Cdk2) protein, a Cdk2 protein and a cell division cycle 25 homolog C (Cdc25C) protein.

11. The pharmaceutical composition according to claim 9, wherein the at lease one cell cycle protein is one selected from a group consisting of a caspase-3 protein, a poly(ADP)ribose polymerase (PARP) protein, a B-cell leukemia/lymphoma x long (Bcl-xL) protein and a B-cell leukemia/lymphoma 2 (Bcl-2) protein so as to induces the cell to go into an apoptosis.

12. The pharmaceutical composition according to claim 9, wherein the flavonoid regulates one of a p38 mitogen-activated protein kinase (MAPK) protein, a c-Jun NH₂-terminal kinase (JNK) ½ protein and a combination thereof so as to induce the cell to go into an apoptosis.

13. The pharmaceutical composition according to claim 9 further comprising a cis-diamminedichloridoplatinum having a structure of Formula II: 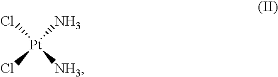
and synergistically functioning with the flavonoid to treat the cancer.

14. The pharmaceutical composition according to claim 1 further comprising a pharmaceutically acceptable carrier.

15. The pharmaceutical composition according to claim 1, wherein the flavonoid inhibits a cancer cell of a mammal.

16. The pharmaceutical composition according to claim 15, wherein the mammal comprises a human being and a rodent.

17. The pharmaceutical composition according to claim 16, wherein the rodent comprises a mouse and a rat.

18. A pharmaceutical composition for treating a cancer, comprising a flavonoid having a first structure of Formula I: 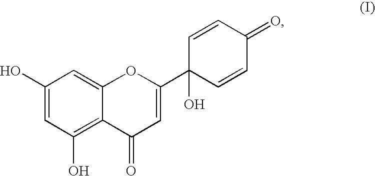
and a cis-diamminedichloridoplatinum having a second structure of Formula II:

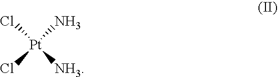

19. The pharmaceutical composition according to claim 18 inhibiting a growth of a cancer cell.
